(19) United States
(12) Patent Application Publication
(10) Pub. No.: US 2023/0340952 A1
(43) Pub. Date: Oct. 26, 2023

(54) SUBMERSIBLE PUMP CONTAMINANT DETECTION SYSTEM

(71) Applicant: INDUSTRIAL FLOW SOLUTIONS OPERATING, LLC, New Haven, CT (US)

(72) Inventor: Mark Grzeika, Thomaston, CT (US)

(73) Assignee: INDUSTRIAL FLOW SOLUTIONS OPERATING, LLC, New Haven, CT (US)

(21) Appl. No.: 18/320,952

(22) Filed: May 19, 2023

Related U.S. Application Data
(63) Continuation-in-part of application No. 17/531,764, filed on Nov. 21, 2021.

(51) Int. Cl. F04B 47/06; F04B 51/00

(52) U.S. Cl. CPC F04B 47/06; F04B 51/00

(57) ABSTRACT

The invention provides an apparatus, system, and method for detecting a layer of contaminants, such as oil and/or hydrocarbons, on the surface of accumulated water within a confined area that is periodically evacuated by a pump. The detection of contaminant layers may be accomplished through the use of an optical detection system comprising a light source, and a conductive sensor comprising a horizontally-disposed plate and a stilling tube. Dissipation of turbulence and agitation of the accumulated water may be achieved by a stilling tube proximate one or more of the sensors disposed within the stilling tube. Additional sensors may detect high, intermediate, and low levels, and an integrated controller may determine the state of media within the confined area, the system being generally configured to keep the oil or other contaminant within the confined area while continuously and/or periodically pumping water away from the confined area.

### CROSS-REFERENCE TO RELATED APPLICATION

This application claims priority to, and the benefit of, co-pending U.S. Non-Provisional patent application Ser. No. 17/531,764, filed on Nov. 21, 2021, entitled “Submersible Pump Detection System”, which is incorporated by reference in its entirety.

### TECHNICAL FIELD

The present disclosure relates generally to the control of pumps or pumping installations specially adapted for raising fluids at a depth and, in particular, to indicating or measuring liquid levels and indicating, measuring, or otherwise distinguishing between various fluid properties within a confined area that is periodically evacuated by a pump.

### BACKGROUND

Submersible pumps and pumping installations serve a variety of common applications. For example, water collection systems often require submersible pumps to avoid overflow and/or to move the water to a larger holding facility or processing station such as, for example, a water treatment filtration plant. Similarly, in low-lying confined areas having important technological infrastructure such as, for example, substations having electrical transformers, submersible pumps are used to protect against environmental and/or other infrastructure damage resulting from unintended flooding of the confined area. In another example, commercial and residential building codes typically require construction of a sump and associated pumping systems for evacuating flood water from the site.

In these applications and others, fluids discharged by submersible pumps may have contaminants in the fluid such as, for example, rain water migrating from a source through the site to the location of the pump. Environmental contaminants including hydrocarbons, oils, and other hazardous substances may be picked up by the rain water and disposed in the moat, or other enclosure, housing the pump associated equipment. Similarly, rainwater runoff in urban areas is subject to contamination from hydrocarbons resulting from oily pavement, rain-washed refuse, or oily spills. The unintended mixture of water with oil represents a common contaminant associated with various applications, and subsequent unabated discharge represents a problem because of the potential harm caused to the environment. A tank farm is one such example where oil and/or other hydrocarbons may inadvertently mix with water at certain locations. Furthermore, governmental regulations dictate and control how and in what condition certain facilities may discharge runoff.

Several control system designs are known that ensure the submersible pump only pumps water out of the confined area, while leaving the oil within the confined area, so that it can be separately discharged, collected, or otherwise removed safely. These systems operate based on the physical properties of water and oil; namely, that water and oil form an immiscible mixture, i.e., an emulsion that tends to separate into distinct layers, where oil tends naturally to rise to the mixture's surface and form a separate layer from the water. As the inlet of a submersible pump is typically positioned toward the bottom of the pump system, the control system will work to maintain the oil layer above the pump's inlet. In this way, the submersible pump system removes the lower water layer as it accumulates, while ensuring that the water level remains high enough to keep the oil layer away from the pump's inlet and within the confined area.

To achieve the desired effect using this arrangement, known control systems employ sensors to detect the presence of water or oil at discrete locations within the confined area. Known solutions may use a mechanical flow switch (e.g., a float) combined with a conductive sensor and/or an optical sensor to operate a submersible pump. For optical sensors, the system may control based on distinguishable reflective properties of the respective media. In terms of conductivity sensors, the system may control based on distinguishable conductivities of the respective media.

However, these systems have proven to be problematic as a level-sensing device in terms of accuracy and/or precision, primarily because external factors promote turbulence, or agitation, of the media as it is deposited within the confined area surrounding the submersible pump. Turbulence may occur due to operation of the pump and/or environmental conditions including wind or rain. Turbulence may also occur based on the geometry of the confined area—as submersible pumps are typically offset below the surrounding area at a distance. This geometry, i.e., by the very nature of the system's geometric configuration, may cause the liquid media to fall into the confined area from a sufficient height to induce mixing and turbulent conditions. The diameter or cross-sectional area of the confined area also impacts the degree to which the media may settle. Conventional systems may further be limited by the amount of contaminant that the system can detect; when turbulence exists, lesser amounts of contaminant may remain undetected, rendering only larger amounts detectable. This means that water levels corresponding to sensor locations where control values must be measured are inherently subject to undesirable conditions as it relates to precise sensor registration and overall system control. Improper accuracy and/or precision of the level-sensing in operations has problems in leaving hydrocarbons undetected and discharged to the environment or otherwise out of the system.

Furthermore, each type of sensor may present certain challenges. For one, mechanical floats/switches are relatively imprecise, as they operate based on a tilting motion, and therefore may trigger the associated electrical relay over a relatively wide water level range. The presence of turbulent water may further affect the precision of a float switch or other level measuring device. For another, conductivity sensors are negatively affected by turbulent conditions, as turbulence promotes mixing, which results in lack of oil detection and/or false readings. Similarly, optical sensors require relatively calm conditions to produce accurate and precise readings. This is because these sensors control, and are calibrated, based on the aforementioned distinguishable physical characteristics of the respective media.

For at least these reasons, a need exists to solve problems for submersible pump applications using known control systems that are subject to inaccurate readings, false alarms, or leaving hydrocarbons undetected and discharged to the environment.

Consequently, a long-felt need exists for submersible pump applications to provide a reliable, precise, and accurate contaminant detection control method of component of a submersible pump system for detecting hydrocarbons and not discharging contaminants outside the system or into the environment. Another long-felt need exists for a control device, system and method that is capable of operating under external conditions that cause turbulence or agitation of the media to be pumped out of the confined area surrounding the pump. Furthermore, a need exists for a modular, replaceable, and easily serviceably control device, system and method for detecting hydrocarbons and not discharging contaminants outside the system or into the environment.

### SUMMARY

The invention according to this disclosure provides an apparatus, system, and method for reliably, precisely, and accurately detecting a layer of a contaminant, like oil, present with water in a confined area that is periodically evacuated by a pump capable of operating under external conditions that cause turbulence or agitation. The apparatus, system, and method for may be configured to detect other contaminants, besides oil, that are of interest.

In one aspect according to the invention, one or more sensors may be located within a stilling pipe, capable of reducing turbulence or agitation to permit desired readings.

In another aspect according to the invention, the stilling tube may be electrically coupled to one or more sensors, the stilling tube being configured to complete a circuit within which different media may be sensed and/or distinguished, based on conductivity or other physical properties of the media. Due to environmental harm and the flammable nature of any hydrocarbon contaminant, care shall be taken and/or safety should be considered in any design or implementation of the present invention to ensure that combustion does not occur of a hydrocarbon or any other flammable contaminant being distinguished from the water.

In another aspect according to the invention, an optical sensor may be disposed proximate a conductive sensor, wherein the optical sensor is adapted to sense the difference between water and air, and the conductive sensor is adapted to sense the difference between water and oil, the combination of sensors being adapted to sense the presence of oil and implement a corresponding control strategy.

In another aspect according to the invention, the apparatus, system, and method is capable of reliably detecting the presence of an oil layer having a thickness of about 1/16 inches (1.59 mm) or thicker. The apparatus, system, and method may be further configured to detect the presence of an oil layer having a thickness of about 1/16 inches (1.59 mm) to about 1/32 inches (0.79 mm). The apparatus, system, and method may be further configured to detect the presence of an oil layer having a thickness of about 1/32 inches (0.79 mm) under ideal conditions.

In another aspect according to the invention, a conductive sensor comprises a sensing plate, an electrically-charged mass such as a stilling tube, and associated circuitry, wherein the conductive sensor is adapted to distinguish between a conductive and a non-conductive liquid.

In another aspect according to the invention, a capacitive sensor is adapted sense and distinguish between dielectric constants of various media, such as air, water, and oil, to selectively perform one or more functions depending on the medium sensed.

In another aspect according to the invention, a control device, system and method using a programmable logic controller (PLC) electrically coupled to one or more sensors, a stilling tube, and/or a sensing plate, the PLC configured to provide reliable, precise, and accurate detection of a layer of contaminants, such as hydrocarbons.

In another aspect according to the invention, the controller circuit is configured to identify a change in a process variable as the input to a proportional-integral-derivative (PID) controller.

In another aspect according to the invention, the apparatus, system, and method includes a variable frequency drive (VFD) electrically coupled to a submersible pump to control the pump over an operable range, thereby reducing the energy required to operate the system.

In another aspect according to the invention, upon detecting a layer of contaminants, the apparatus, system, and method uses a controlled activation of one or more alarms to indicate the presence of the contaminants, pump failure, system component failure, or exceedingly high media level(s). Another aspect is to use a solid-state contactor for the controlled activation of alarms for the indicators monitored by the apparatus, system, and method.

In another aspect of the present disclosure, the apparatus, system, and method provides remote monitoring capability using cellular cloud-based monitoring, local network monitoring, and/or dry contact monitoring.

In yet another aspect of the present disclosure, upon detecting a layer of contaminants, the apparatus, system, and method controls a submersible pump so that the layer of contaminants remains above the pump inlet, while simultaneously allowing water to be evacuated from the confined area.

### DESCRIPTION OF DRAWINGS

Non-limiting and non-exhaustive embodiments of the present disclosure are described with reference to the following drawings. In the drawings, like reference numerals refer to like parts throughout the various figures unless otherwise specified.

For a better understanding of the present disclosure, reference will be made to the following Detailed Description, which is to be read in association with the accompanying drawings, which are incorporated in and constitute a part of this specification, show certain aspects of the subject matter disclosed herein and, together with the description, help explain some of the principles associated with the disclosed implementations, wherein:

FIG. 1 is an elevation view of a submersible pump detection apparatus, system, and method, in accordance with an embodiment of the present invention;

FIG. 2 is an enlarged view taken along the line shown in FIG. 1, in accordance with an embodiment of the present invention;

FIG. 3A is a schematic elevation view of an optical sensor assembly illustrating an air condition, in accordance with an embodiment of the present invention;

FIG. 3B is a schematic elevation view of an optical sensor assembly illustrating an oil condition, in accordance with an embodiment of the present invention;

FIG. 3C is a schematic elevation view of an optical sensor assembly illustrating a water condition, in accordance with an embodiment of the present invention;

FIG. 4 is a schematic diagram of signal processing circuitry for a submersible pump detection system, in accordance with an embodiment of the present invention;

FIG. 5A is a table indicating conductive and optical sensor capabilities of distinguishing different exemplary media, in accordance with an embodiment of the present invention;

FIG. 5B is a graph corresponding to a capacitance sensor and two distinct events, E₁ and E₂, in accordance with an embodiment of the present invention; and

FIG. 6 is an elevation view of a submersible pump detection apparatus, system, and method, in accordance with an alternative embodiment of the present invention.

### DETAILED DESCRIPTION

Non-limiting embodiments of the invention will be described below with reference to the accompanying drawings, wherein like reference numerals represent like elements throughout. While the invention has been described in detail with respect to the preferred embodiments thereof, it will be appreciated that upon reading and understanding of the foregoing, certain variations to the preferred embodiments will become apparent, which variations are nonetheless within the spirit and scope of the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “some embodiments”, “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are provided for the purposes of illustrating some embodiments of the present disclosure, and are not to be considered as a limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

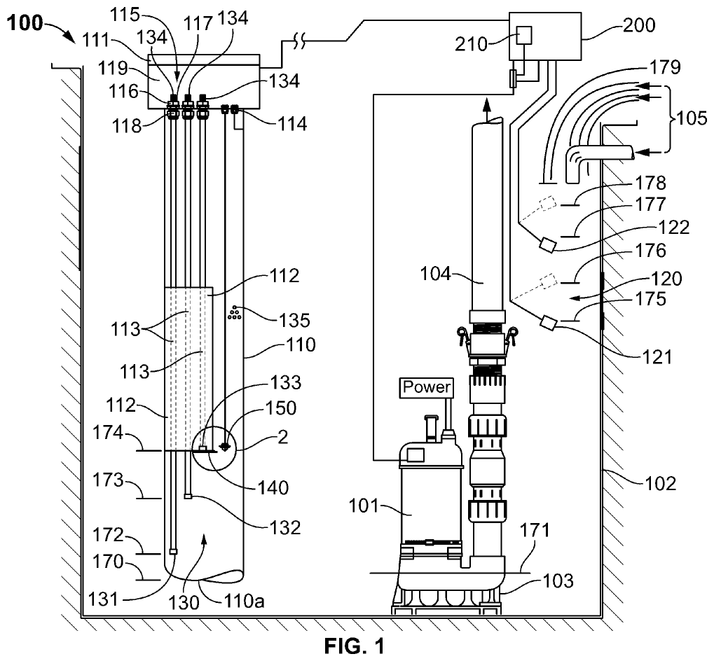

The submersible pump detection apparatus, system and method 100 according to the present invention can be incorporated into a wide range of pumping installations and applications, can use other components and arrangements to achieve similar objectives, and is described in the context of a detection submersible pump detection system used in contaminant application as an exemplary embodiment as shown in FIGS. 1-5. The submersible pump detection apparatus, system and method 100 may use suitable sensors including capacitive sensors, pressure sensors, optical sensors, and ultrasonic sensors. While the invention is described in this disclosure within the context of oil detection applications, in particular oil having a layer thickness of about 1/32 inches (0.79 mm), other hydrocarbons are contemplated as falling within the scope of this disclosure. Similarly, any other contaminant that has a detectable electrical property capable of being sensed and distinguished from air and/or water falls within the scope of this disclosure; e.g., conductance (Siemens) capacitance (Farad, or dielectric constant). In any design or implementation of the present invention, care shall be taken to ensure combustion does not occur of any hydrocarbon or other flammable contaminant.

FIG. 1 is a schematic elevation view of an exemplary preferred embodiment of a submersible pump detection system 100, according to the present invention, having particular utility in a confined area and in conjunction with a pump capable of evacuating liquid media from the confined area using various system components such as piping, according to the present invention. In this context, the term “confined area” may refer to any defined area that may contain fluid media and which may be evacuated by an active process such as pumping, including but not limited to sumps, moats, transformer vaults, elevator shafts, and other equivalent structures. Pumps typically employed for this purpose include, but are not limited to, submersible pumps and suction pumps. Furthermore, the submersible pump detection system 100 may be configured to control a single pump, or multiple pumps that serve the purpose of providing pump diversity, redundancy, or other similar purposes. For ease of illustration, FIG. 1, and subsequent depictions, show a single pump, although the submersible pump detection system 100 may control more than one pump

As is illustrated in FIG. 1-4, a submersible pump detection apparatus, system, and method is generally shown as element 100. The pump detection system 100 is configured to be installed with a pump 101 and operated within a confined area 102 purposed to evacuate or otherwise pump accumulated water through pump inlet 103, located proximate a pump inlet level 171, through to discharge tube 104, while detecting the presence of a contaminant, like hydrocarbons, that may intermix with the accumulated water. As demonstrated here, the accumulated water may enter the confined area 102 through a confined area supply 105, which may be a pipe, grate, or any other mechanism that conveys accumulated fluids, liquids, and/or water from a source to the confined area 102. Advantageously, as will be elaborated upon below, the pump detection system 100 detects or senses the presence of contaminants regardless of how turbulent or agitated the accumulated water may be within the confined area 102.

In one aspect of the present invention, pump detection system 100 may include a stilling tube 110, which may be configured to calm or otherwise dissipate turbulent conditions of the accumulated water within the confines of the stilling tube 110. To achieve this effect, a stilling tube inlet 110a may be located adjacent the lower portion of the confined area 102, as shown in FIG. 1, which is depicted as stilling tube inlet level 170. Level 170 is positioned, vertically with respect to the ground, and laterally with respect to the walls of confined area 102, so that it may accept accumulated water within the interior, but provide for turbulence-dissipating effects as quickly and effectively as possible. Depending on the application, stilling tube inlet level 170 may be positioned below, at, or above, the pump inlet level 171. Similarly, the geometry of stilling tube 110 may take any form to achieve the desired effect; for example, the cross-section of stilling tube 110 may be circular, square, rectangular, or elliptical. The cross-sectional area of stilling tube 110 may remain constant along its length, or may be tapered. Also, the cross-sectional area of stilling tube 110 may transform shape along its length such that one end takes the form of a first shape, such as a square, and the other end takes the form of a second shape, such as a circle. Additionally, stilling tube 110 may be formed of any thickness, uniform or tapered; in a preferred embodiment, the stilling tube 110 is uniformly ¼ inch thick.

Stilling tube 110 may further be configured with a purge opening located at an upper end thereof (not shown), to negate pressure effects of air contained within the interior of the stilling pipe 110. The purge opening would therefore allow air to enter and exit the stilling pipe as the accumulated water level changes, so that a pressure differential does not occur across the stilling pipe boundary, allowing the internal level and external level of the accumulated water to remain the same. The purge opening may alternatively be formed by the cumulative effect of openings provided by the system components disposed atop the stilling pipe 110. Additional openings disposed along portions of the stilling tube 110 may be included. For example, one or more openings 135 may be disposed along and through the body of the stilling tube 110 to allow for migration of fluids through the pipe along a portion of the side thereof, alternatively or in combination with, stilling tube inlet 110a. As shown in FIG. 1, openings 135 are representatively shown in a localized area on the surface and extending through stilling tube 110, for illustrative purposes. Openings 135 may be of any shape that allows for migration of fluids through the stilling tube 110 while also being conducive with minimizing fluid disturbance, surface ripples, etc. In one embodiment, for example, the openings 135 may be formed as a strip extending from proximate stilling tube inlet 110a to proximate purge opening at the top end of the stilling tube 110.

Furthermore, the stilling tube 110 may be configured to house one or more sensors 130, and/or one or more optical sensors 150 therein. As such, the cross-sectional area, as detailed above, may be further sized in any manner suited to achieve the dissipation of accumulated water, the housing of said one or more sensors 130, the housing of said one or more optical sensors 150, and the space required for the pump detection system 100 to operate reliably, accurately, and precisely. As further detailed below, the one or more sensors 130 and/or the one or more optical sensors 150 may be configured to reliably, accurately, and precisely detect the presence of contaminants, like hydrocarbons, present within the confined area 102. In this context, the term “accuracy” may refer to how closely individual measurements agree with the correct, or true, value. Also in this context, the term “precision” may refer to a measure of how closely individual measurements agree with one another. Furthermore, in this context, the term “reliability” may refer to a level of confidence in detecting contaminants based upon the acceptable calibration of the apparatus and/or system, and based upon the repeatability of the measurements obtained. As mentioned, an objective of the stilling tube 110 is to promote an immiscible mixture to form (i.e., an emulsion of, for example a hydrocarbon layer on top of a water layer). This is largely achieved by separating the general accumulated water within the confined area from the localized accumulated water adjacent the sensors 130, 150, so that the localized water may settle, turbulent effects may substantially dissipate such as, for example, settling the mixture from foam to a non-emulsion mixture, and sensors 130, 150 may accurately and precisely register a reading of the conditions to determine whether contaminants are present.

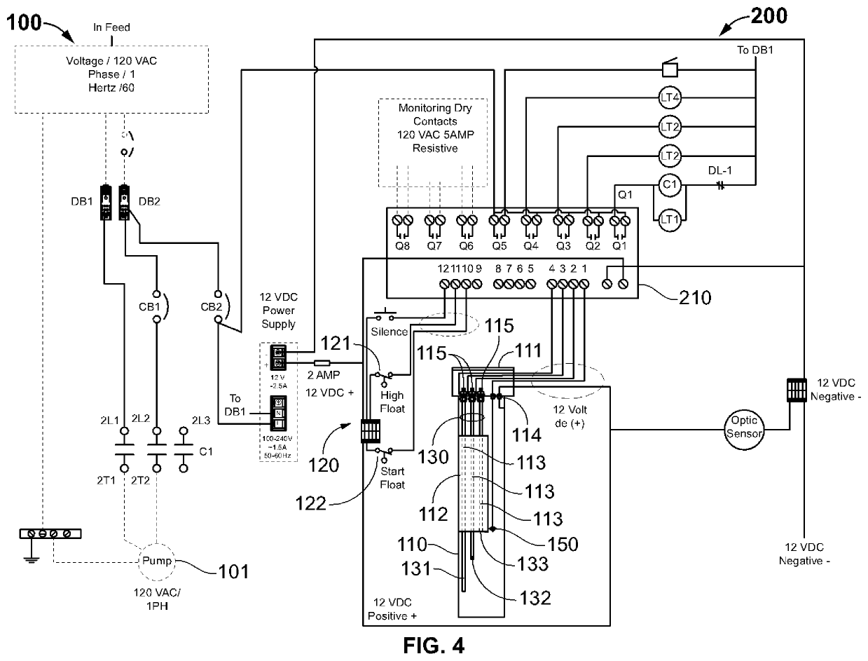

Stilling tube 110 may be further configured to electrically couple to a control module device 200. Stilling tube 110, therefore, may further facilitate registration and readings by forming at least a portion of one or more sensors 130 in the context of stilling tube 110 forming part of a circuit to sense the conductance of one or more media within the stilling tube 110. For example, in a preferred embodiment, stilling tube 110 is biased at +12V DC relative to the one or more sensors 130 via an isolated contact 114 connected to the stilling tube 110, as shown in FIGS. 1 and 4. Stilling tube 110 therefore may be made of a conductive material, including but not limited to, stainless steel, aluminum, zinc, and brass. Empirical studies conducted by Applicant have demonstrated that the mass of the stilling tube 110 may facilitate proper registering of a low voltage current through the medium, and therefore the stilling tube 110 may be well suited for multiple purposes, for example, when a conductivity sensor is employed. Furthermore, the stilling tube 110 may comprise more than one material, so that only a portion of the stilling tube 110 is used for registering a physical property/value.

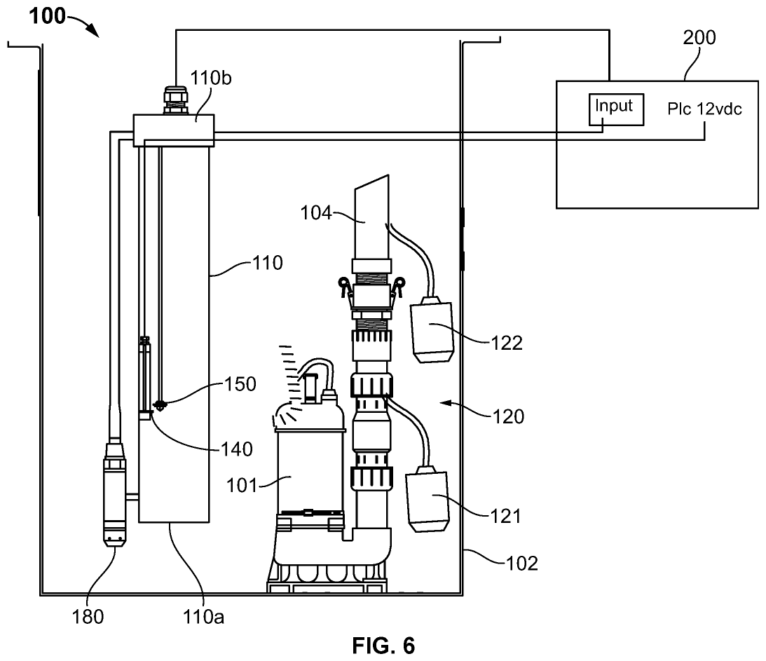

In another aspect according to the present invention, as illustrated in FIGS. 1-6, stilling tube 110 may contain, within an interior space, one or more sensors 130. Sensors 130 may include first sensor 131, second sensor 132, and third sensor 133, disposed within stilling tube 110 according to the arrangement depicted in FIG. 1. Sensors 130 are disposed within stilling tube 110 in manner that allows for reliable, accurate, and precise registering of readings of one or more physical properties of media contained therein. In a preferred embodiment, sensors 130 are electric conductivity sensors. In an alternative embodiment, capacitive sensors 130 are contemplated. In yet another alternative embodiment, first and second sensors 131, 132, comprise a level transducer, for example, a continuous output level sensor, as shown in the embodiment of FIG. 6.

In the context of the preferred embodiment and with reference to FIGS. 1-5A, conductivity sensors 130 may be configured to distinguish between water and oil. The conductivity sensor may be made intrinsically safe to meet international standards for hazardous locations, such as, for example, a tank farm, chemical tank, a non-grounded well, and other hazardous applications. First sensor 131 may be disposed at a first probe level 172, and may be configured and/or calibrated to sense a condition that prevents contaminants from entering pump inlet 103 by maintaining the accumulated water level above pump inlet level 171. Second sensor 132 may be disposed at a second probe level 173, and may be configured and/or calibrated to sense a condition that turns the pump 101 on, in the condition that a contaminant has previously been identified, so that, under operating conditions having an identified contaminant, the level at its lowest for that condition so as to mitigate the likelihood of adverse conditions such as turbulence from causing contaminant to be sucked in to the inlet 103, such as may occur due to turbulence at the first probe level 172. Third sensor 133 may be disposed at a third probe level 173. Third sensor 133 may be configured to detect the presence of oil, or other contaminant of distinguishable electrical property. Third sensor 133 may be electrically coupled to sensing plate 140 to facilitate detection.

Sensing plate 140 may be configured to provide a bottom surface upon which may be adapted to register a reading by sensing the relative conductance of a media located at that level with respect to the horizontal, i.e., horizontally within the gravity field and/or planar surface or layers of liquid within the stilling tube 110. In accordance with this objective as shown in FIGS. 1-6, sensing plate 140 comprises a substantially flat lower surface disposed at a lower surface level 174. According to one principle of operation the sensing plate 140 need only become saturated with a discrete, continuous layer of liquid thereon, as the liquid is drawn down by the pump from a liquid level occurring above the lower surface level 174.

The one or more sensors 130 may further be coupled to, and insulated from, the stilling tube 110 wall by a PVC mounting block 112. The mounting block 112 may be fitted with probe holes (rods) 113, which provide a press fit for the sensors 130. Electrically isolated feed-through assemblies 115 may be disposed at the top of the stilling tube 110 for each of the sensors 130. Additional system components, as shown in FIG. 1, may include: one or more insulated conductors 114, connected to said stilling tube 110; removeable access cover 111; nut 116; washer 117; grip connector 118; cable conductor 119; and threaded ends 134, to which sensors 130 may be affixed and ensure electrical coupling to the other parts of the pump detection system 100.

Under normal operating conditions, the pump will run when the start float 121 is activated and stop when the water level is pumped to the third sensor (stop probe) 131. Under adverse conditions, such as wind and rain, the surface of the water in the confined area 102 may be disturbed and deviate significantly from steady-state levels/conditions, causing ripples on the liquid surface and mixing of layers therein. Under such conditions, the accumulated water level sensing accuracy and precision would become compromised, but for the advantageous arrangement as substantially described and shown herein.

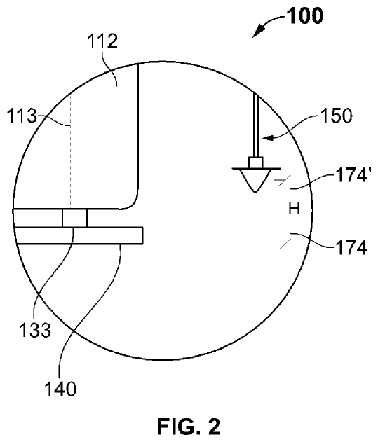

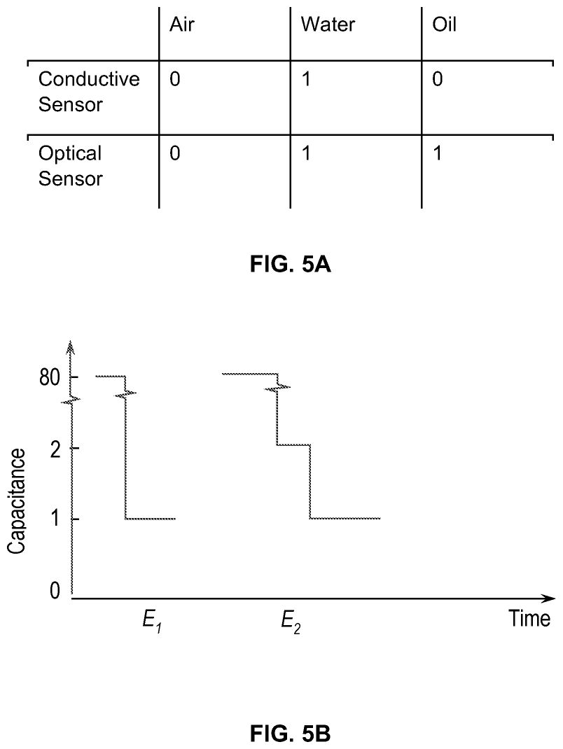

As shown in FIGS. 1-6, pump detection system 100 may comprise one or more optical sensors 150. Turning now to the enlarged view FIG. 2, taken from FIG. 1, certain particulars of an embodiment of pump detection system 100 may be seen. In general, optical sensor 150 is purposed to differentiate between water and air, as shown in table of FIG. 5A; once a filling condition occurs in the confined area 102, the water level will rise above optical sensor level 174′—in operation, as will be further elaborated upon below, as the liquid level is drawn down, optical sensor will engage conductivity sensor 133 upon detection of air at level 174′. Furthermore, optical sensor 150 may register a reading at an optical sensor level 174′, which may be offset from sensing plate lower surface level 174′ of sensing plate 140, i.e., the level that sensor 133 may register a reading, and this vertical offset distance is denoted as H in FIG. 2. In a preferred embodiment, H equals zero (0) inches, which is to say that the optical sensor engages conductivity sensor 133 to register a reading simultaneously. Such an arrangement may simplify the control logic of the system, wherein concurrent and/or simultaneous reading are registered by sensors 133 and 150. However, H may be selected at any distance, positive or negative (i.e., below or above one another). For non-zero H values, two variables effect the control logic and/or time sampling required: (i) the rate of liquid draw down, R, provided by the pump and (ii) the offset distance H. For confined areas 102 having constant cross-sectional area and constant-speed pumps, the time delay ΔT=H/R. For a cylindrical confined area 102, R=πr²*|*p, where: r=radius of the confined area 102; |=height of the liquid volume to be emptied; and p=pump speed, such as in liters per second. Therefore, for offset, non-zero H values, dynamic measurement characteristics need to be taken into consideration to register correct oil detection, and in general, the system would be calibrated based upon a specific draw down rate, R. Also shown in FIG. 2, third sensor 133 is shown horizontally offset from optical sensor 150; here, the two sensors need not interfere with one another spatially and/or with respect to liquid drawdown.

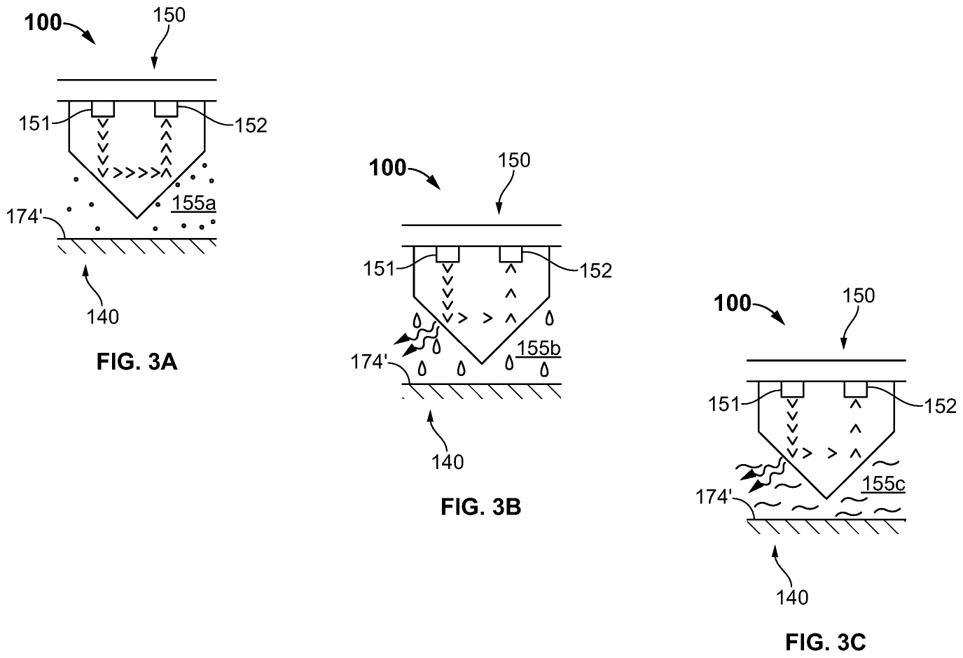

Turning now to FIGS. 3A-3C, the one or more optical sensors 150 are shown in various operating conditions indicative of the sensing condition contemplated within this disclosure as conditions typically observed by pump detection system 100. Common to FIGS. 3A-3C, one type of optical sensor 150 is shown having a transmitter 151 and a receiver 152, contained within a translucent housing. The translucent housing may take any shape configured to transmit, reflect, and receive an optical signal capable of calibrating based on unique properties of media, including but not limited to pyramidal and prismatic shape. Utilizing this configuration, the optical sensor 150 may be substantially saturated by the surrounding media, or medium, as depicted in FIGS. 3A-3C. In each of these illustrations, an optical signal is irradiated from the transmitter, a certain percentage is absorbed into the translucent housing, a certain percentage is transmitted through the surrounding medium, and a certain percentage of the optical signal will be reflected back to the receiver, which ultimately varies as a function of the surrounding medium's emissivity/absorbance. In effect, in an oil or water condition as in FIGS. 3B or 3C, light dispersion reduces the amount of light received back to the receiver 152 via dissipation or refraction through the oil or water. In contrast, less or no dispersion or refraction occurs in the air medium, as illustrated in FIG. 3A. This principle therefore allows optical sensor 150 to distinguish between air and a liquid medium.

As illustrated in the table of FIG. 5A, the combined functionality of the conductivity sensor 133 and optical sensor 150 provides that a true value is indicated as “1” and a false value is indicated as “0”. For the optical sensor, a liquid such as either water or oil registers a “1”, and air registers a “0”, according to the principle just described. For the conductivity sensor, however, only water registers a “1”, i.e., based upon its conductive nature, whereas air or oil registers a “0”, based upon their non-conductive nature. In this way, upon drawdown or other configurations/system operations, optical sensor 150 may first determine the presence of water or oil, then determine the presence of air, thereby engaging the conductivity sensor 130, in particular third sensor 133. Due to the calming nature of the stilling tube 110, any oil will have settled on the surface of the water, and the conductive sensor can thereby distinguish or discern whether oil “0” is present, or water “1”. In this way, the combination of conductivity sensor 133 and optical sensor 150 reduce error and promote precision, accuracy, and reliability.

Referring to FIG. 5B, in an alternative embodiment according to the present invention employing one or more capacitive sensors 130, such as a third capacitive sensor 133, a capacitive sensor may be able to discern between three separate media, including water, air, and oil based on the dielectric constants of each respective medium. For example, capacitive level sensors operate on the property that the liquid is known as the dielectric using the key property of dielectric materials and the dielectric constant or relative permittivity with air having a dielectric constant of: air=1 (at STP, for 0.9 MHz, capacitance is 1.00058986±0.00000050); e.g. diesel or transformer oils have a dielectric constant range=2.1 to 2.5; and water=80 (ranging from 80.1 at 20° C. to 10 at 360° C.). According to a first event E₁, corresponding to liquid drawdown relative to third capacitive sensor 133, sensor 133 initially detects water at a capacitance of about 80, followed by a distinct drop and subsequent registration of air at a capacitance of about 1. Here, the system 100 may remove all or substantially all of the liquid from confined area 102, because no oil was sensed. Conversely according to a second event E₂, also corresponding to liquid drawdown relative to third capacitive sensor 133, sensor 133 initially detects water at a capacitance of about 80, followed by a distinct drop and subsequent registration of oil at a capacitance of about 2, followed by another distinct drop and subsequent registration of air at a capacitance of about 1. In this way, the third capacitive sensor 133 may be used independently with the calming effects of the stilling tube 110, or alternatively, third capacitive sensor 133 may be used in combination with an optical sensor 150 for initiating, i.e., turning on, third capacitive sensor 133, and/or in combination with other sensors, e.g., sensors 131, 132 and/or others. Applicant has contemplated certain limitations of capacitive sensors, e.g., third capacitive sensor 133, as used and described herein, which primarily pertain to fluid dynamics. For example, to gain an accurate reading, a capacity sensor should contain within each capacitive plate a single medium. As the distance between said plates reduces, fluid dynamics effects, such as adhesion of medium to the surface of one or both plates thereof, to take effect. The result may be the inadvertent, and unwanted presence of a meniscus layer of one medium along at least a portion of the plate and the presence of a second and/or third medium also physically located in between the plates. As the layer to be sensed and the corresponding distance between the capacitive plates reduces to 1/16 inches, or 1/32 inches and below, one skilled in the art may take appropriate measures to ensure accurate functioning of the sensor. Horizontal and/or vertical orientations of the plates may each have certain desirable and/or undesirable effects.

In another aspect of the present invention, pump detection system 100 may include a control module device 200, as shown in FIGS. 1, 4, and 6. As will be further detailed below, control module device 200 may be electrically coupled to one or more system components to achieve one or more of the operations contemplated in the present disclosure, including but not limited to: evacuating accumulated water; detecting contaminants, such as hydrocarbons; retaining contaminants by elevating and maintaining the accumulated water level above the pump inlet level 171, while simultaneously evacuating additionally accumulated water via discharge tube 104; turning on, off, or otherwise modulating the speed of the pump 101; enacting audible/visual alerts; and/or communicating with wireless network and remote control systems.

In the embodiment shown in FIG. 4, the control module device 200 comprises a 12V DC PLC 210 with sensing inputs and actuating outputs. The inputs may include a start float 121, a high-level float 122, first, second, and third sensors 131, 132, 133, respectively, and one or more optical sensors 150. For a single float 120 embodiment, the float functions as a start float 121. In an optional float 120 embodiment, high level float 122 may be added for sensing an imminent overflow condition. The outputs include pump on/off relay, one or more audible alerts, and/or one or more visual indicators, for notifying the presence of oil, a high-water level, and/or that the pump 101 is running as expected. As illustrated in FIG. 1, control module device 200 may be electrically coupled to the stilling tube 110, one or more sensors 130, one or more optical sensors 150, pump 101, and mechanical floats 120 (including start float 121 and high-level float 122). In operation, the start float 121, upon sensing an imminent overflow condition, may be configured for controlling the starting of the process to discharge water from the confined area 102, under control of the pump detection system 100, and the high level float 122 can be used for other control functions or as a confirmation signal for the starting the process to discharge water, if desired.

As shown in FIG. 6, in an alternative embodiment according to the present invention, the control module 200 may use an input signal from a pressure transducer to accurately monitor the level such as, for example, 4 to 20 milliamp pressure transducer. When a thin layer of hydrocarbons passes through the detection area, a PLC 210 may send a signal to a third sensor 133 VFD may be disposed at a third probe level 173 to maintain a level that would be considered safe so oil cannot enter the suction area of the pump this is accomplished through a PID function on the drive itself.

Certain operating modes of the various embodiments contemplated herein are now described, in accordance with the present invention. Throughout all operating conditions, the stilling tube 110 is sized, positioned, and configured, e.g., with one or more openings 135, to maintain a level liquid surface—irrespective of the level of the liquid or geometric—under all external conditions. The stilling tube may be configured to sense the liquid level at three points, i.e., height levels: the stop level, i.e., first probe level 172; an intermediate stop level, i.e., second probe level 173; and an oil-sensing level, i.e., third probe level 174. These correspond to first, second, and third sensors 131, 132, and 133, respectively. Generally, sensors 131, 132, 133 may detect differences in physical properties, e.g., conductance, capacitance, between water and/or oil, and/or air, thereby determining the presence of oil. The stop probe 131 may be set at a level, first probe level 172, that is proximate and above said pump inlet level 171, which ensures contaminants do not pass within pump inlet 103 of the pump 101. As previously mentioned, under normal operating conditions, the pump will run when the start float 121 is activated and stop when the liquid level is pumped to the third sensor, stop probe, 131. This provides the proper conditions for oil detection of the various embodiments described herein, wherein drawdown of the liquid level, generally, furthers the calming and settling characteristics of the liquid, such that accurate reading may be registered by the sensors, e.g., optical sensor 150, third sensor 133.

In a first mode of operation, liquid accumulation begins, via e.g., a rain event, and liquid fills confined area up to start probe 121, wherein the liquid level moves start probe 121 from start float OFF level 175 to start float ON level 176. At this point, if for whatever reason liquid further advances to a higher level, high-level sensor 122 will sense the condition by moving from high float OFF level 177 to high float ON level 178, and thereby indicate via, .e.g., an alarm, that the system is overloaded and/or flooding. However, under normal operating conditions the liquid level may be drawn down and/or expelled until the liquid passes either or both the optical sensor 150 and the third sensor 133, upon which the aforementioned oil detection cycle initiates. Upon detection of no oil, i.e., water-only conditions or contaminant-free conditions, the liquid level may be drawn down and/or expelled either preferably to first probe level 172, or alternatively to pump inlet level 171, to the conclusion of the operating mode wherein the detection system may be reset for the next cycle, e.g., a different liquid accumulation within the confined area 102.

In a second mode of operation, liquid accumulation begins, via e.g., a rain event, and liquid fills confined area up to start probe 121, wherein the liquid level moves start probe 121 from start float OFF level 175 to start float ON level 176. High-level sensor 122 may or may not come in to play as in the first mode of operation. Then, the liquid level may be drawn down and/or expelled until the liquid passes either or both the optical sensor 150 and the third sensor 133, upon which the aforementioned oil detection cycle initiates. Upon detection of oil, i.e., water with contaminant conditions detected, the liquid level may be drawn down and/or expelled to the second probe level 173 of second sensor 132, thereby maintaining oil or other contaminant within the confines of the confined area 102. In this second mode of operation, any of the embodiments described with respect to the manner in which oil detection may be achieved are applicable.

In other modes of operation, certain alarm functions may be employed. For example, in a scenario in which oil detection cycles via optical sensor 150 and third sensor 133 do not detect water through the drawdown stage, but instead only detect oil, or other contaminant, then an alarm may be employed to alert personnel that the system may be overrunning with contaminant. As another example, alarms associated with high level detection vis-à-vis high float 122 may signal general system overrun. As another example, failure to detect during cycling may initiate an alarm to indicate potential failure of one or more sensors and/or other mechanisms needing inspection. As another example, if a conductive condition is detected vai the optical sensor 150 simultaneously, or within an acceptably calibrated/tolerated time delay ΔT, giving the indication of oil being present, and a signal is not present at input 5, so as to turn off the PLC 150 due to loss of conductivity between the stilling tube 110 and the oil probe protruding plate 140, then the system may provide a hydrocarbon alarm to indicate the presence of a contaminant.

The proper functioning of the sensors 130 generally requires that a homogeneous layer of liquid in between the stilling tube 110 and instant sensor 130, e.g., third sensor 133. Thus, for a thin layer of hydrocarbons to be properly detected, the calming effects of the stilling tube 110 are necessary to achieve the desired result in many field applications.

FIG. 4 illustrates a detailed schematic diagram of exemplary physical property sensor and actuation circuitry, in accordance with submersible pump detection system 100 that uses a physical property sensor in a manner that can detect oil when the level is rising through the detection area or being pumped down through the detection area. Those skilled in the art will understand that in other embodiments, the signal processing can be partially or entirely contained and executed within the software of a microcontroller or other circuitry to achieve similar functionality as that identified herein; therefore, such alternatives do not depart from the scope of this disclosure.

In an alternative embodiment in combination with any of the above, a variable speed (VFD) pump and/or PID controller may be added to allow for a continuous liquid level maintenance mode wherein the water level is maintained within a small range. In another alternative embodiment in combination with any of the above, communications hardware may be added to the system to effect internet connectivity for remote monitoring and/or control. The communications hardware may take the form of, for example, an ethernet adapter, a Wi-Fi adapter, or a cellular or other radio frequency transceiver. And, in an alternative embodiment in combination with any of the above, the system may be fitted with solar power and battery backup.

The previous description of the disclosed embodiments is provided to enable any person skilled in the art to make or use the invention. Various modifications to these embodiments will be readily apparent to those skilled in the art, and the generic principles defined herein can be applied to other embodiments without departing from the spirit or scope of the invention. It is therefore desired that the present embodiments be considered in all respects as illustrative and not restrictive, reference being made to the appended claims as well as the foregoing descriptions to indicate the scope of the invention.

What is claimed is:

1. A submersible pump detection apparatus comprising:
a sensor in electric communication with a submersible pump, said sensor configured to detect a level of a fluid within a confined area; and
a stilling tube having a first opening disposed above a second opening, and a middle portion disposed therebetween, said sensor being disposed within said middle portion, wherein said stilling tube is configured to stabilize the fluid within an area proximate said sensor so that said sensor can register an accurate reading of a property of the fluid.

2. A modular sensing unit comprising:
first, second, and third sensors, wherein each sensor is adapted to sense a property of one or more media at a distinct location, each sensor being disposed at a separate location to sense said one or more media and being at least partially embedded within an insulating block,
wherein said sensors further comprise a unitary device having plug-and-play connection, adapted to be installed and/or removed within a pump detection system.
